(19) United States
(12) Patent Application Publication
(10) Pub. No.: US 2023/0149736 A1
(43) Pub. Date: May 18, 2023

(54) LIGHT-BASED DENTAL TREATMENT DEVICE

(71) Applicant: DENTOSITY, LLC, Solon, IA (US)

(72) Inventors: Kimberly MORIO, Solon, IA (US); Kim A. BROGDEN, Coralville, IA (US); Robert STERNOWSKI, Cedar Rapids, IA (US)

(73) Assignee: DENTOSITY, LLC, Solon, IA (US)

(21) Appl. No.: 18/155,388

(22) Filed: Jan. 17, 2023

Related U.S. Application Data
(63) Continuation of application No. 17/201,842, filed on Mar. 15, 2021.
Application No. 17/201,842 is a continuation of application No. PCT/US20/55065, filed on Oct. 9, 2020.
(60) Provisional application No. 62/913,831, filed on Oct. 11, 2019.

(51) Int. Cl. A61N 5/06; A61C 1/00; F21V 8/00; A61L 2/00

(52) U.S. Cl. CPC A61N 5/0624; A61C 1/0046; A61N 5/062; G02B 6/0006; G02B 6/0008; A61L 2/0047; A61C 5/50

(57) ABSTRACT

A light source for emitting light, a light conduit in electromagnetic communication with the light source for communicating light from the light source to the area of interest in three dimensions, and a controller combined to the light source for controlling the intensity of the light emitted to the area of interest.

### CROSS-REFERENCE TO RELATED APPLICATIONS

This application is a continuation of U.S. patent application Ser. No. 17/201,842 filed on Mar. 15, 2021, which is a continuation of international patent application PCT/US2020/055065, filed on Oct. 9, 2020 designating the U.S., which claims the benefit of U.S. Provisional Patent Application No. 62/913,831 filed Oct. 11, 2019, all of which are incorporated herein by reference.

### TECHNICAL FIELD

This document pertains generally, but not by way of limitation, to treatments and instruments for treating microorganisms within and around the oral cavity and within and around anatomical passages or cavities.

### BACKGROUND INFORMATION

Endodontic treatment includes, in part, bacterial disinfection of a root-canal system and the prevention of re-infection. In some examples, endodontic treatment involves chemical and mechanical debridement of the canal space for disinfection. Chemical irrigation infiltrates the root-canal system, and disinfects or dissolves tissue and removes necrotic debris from the canal wall.

For instance, irrigation of the canal system after mechanical formation of a passage removes tissue remnants, microorganisms and dentin chips by a continual flushing of the canal space. A combination of irrigants in sequence is optionally used for treatment. One example of an irrigant includes sodium hypochlorite (NaOCl) for its efficacy for disinfection and ability to dissolve organic material. In other examples, sodium hypochlorite is used in combination with Ethylenediaminetetraacetic acid (EDTA). The addition of chlorhexidine (CHX) as an irrigant is also used in some example because of its antimicrobial activity, for instance against Enterococcus faecalis (E. faecalis).

A problem to be solved includes enhancing the disinfection of the canal system (or other anatomical passage or cavity). The instrumentation of the canal space is a step in the process of cleaning and disinfection. Mechanical instruments have limitations due to the complexity of the canal systems (e.g., lateral canals, fins and crevices along canal walls or the like). This has been demonstrated by microcomputed tomography (CT) scanning which showed large areas of the root canal walls that were left untouched by instruments. The instruments have limited ability to navigate the canal space and reach tissue remnants, microorganisms and dentin chips retained in these tortuous spaces. Accordingly, the clinician is reliant on the chemical irrigation of the canal system to disinfect the untouched canal features and achieve a successful outcome. However, chemical irrigants are also subject to the tomography of the canal (e.g., lateral canal passages, crevices, fins or the like) and in some examples fail to disinfect features of the canal. For instance, the flushed chemical irrigants fail to adequately reach tortuously hidden features along or extending from the canal. Additionally, remnant tissues, microorganisms or the like are, in some examples, suspended in or concealed by biofilms, collections of proteins, carbohydrates or the like that further complicate access by irrigants.

Accordingly, there is a need for an improved light based dental treatment device.

### SUMMARY

In accordance with one aspect of the present invention, there is disclosed is a device for disinfecting an area of interest. The device comprises of a light source for emitting light, a light conduit in electromagnetic communication with the light source for communicating light from the light source to the area of interest, and a controller combined to the light source for controlling the intensity of the light emitted to the area of interest. The light conduit comprises a cylindrical distal end and wherein light radiates from the cylindrical distal end in three dimensions. In this regard, the light conduit comprises of a transverse core that channels light from the light source therethrough and a cladding layer surrounding the core to the beginning of the cylindrical distal end to block light from exiting the core before reaching the cylindrical distal end.

In one implementation, the cladding layer is removed from the light conduit at the cylindrical distal end where the cylindrical distal end is a portion of the core of the light conduit without the cladding layer. The light conduit can be a plastic fiberoptic conduit comprising a cladding that has been removed from a distal end so that light is emitted radially and axially relative to the distal end of the conduit for insertion into a volume of interest to treat the affected area of interest.

In yet other implementations, the controller comprises of a constant current device for controlling the current to the light source throughout the temperature range of the light source. The constant current device can have a milliamp current to the constant current device, which is reducible by an analog voltage or a pulse-width modulated signal to the constant current device, and where the light source is a light emitting diode and the constant current device provides a constant current to the light emitting diode throughout the temperature range of the light emitting diode. The controller can comprise of a duty cycle controller to modify a pulsating on/off rate of the light source.

In other implementations, the light conduit comprises of a branch connected to an irrigant channel for communicating an irrigant into the light conduit for simultaneous application of the irrigant and the light to the area of interest. An irrigant source can be connected to the irrigant channel for supplying the irrigant; a selectively operated cover that selectively stops the flow of irrigant into the light conduit; and a pump for forcing irrigant into or suctioning irrigant out through the channel. In such implementations, the irrigant is one chose from bleach, saline, and water. With bleach, the UVC light activates the bleach by releasing free radicals from the bleach in the light conduit prior to communication of the irrigant to the area of interest.

The light source can be a UVC light emitting diode. A lens can be positioned between the light source and the input of light conduit for focusing the light from light source into light conduit. The housing can house the light source, the controller, and the lens. The attachment mechanism selectively attaches the light conduit to the housing with the light conduit aligned with the lens to focus light from the light source into the light conduit.

### BRIEF DESCRIPTION OF THE DRAWINGS

These and other features and advantages of the present invention will be better understood by reading the following detailed description, taken together with the drawings wherein:

FIG. 1 illustrates, by way of example, a perspective view diagram of an embodiment of an electromagnetic energy-based tissue treatment system.

FIG. 2 illustrates, by way of example, another perspective view diagram of an embodiment of the electromagnetic energy-based tissue treatment system.

FIG. 3 shows the device of FIG. 1 from the front. FIG. 4 shows section A-A from FIG. 1.

FIG. 5 shows an electrical schematic for device.

FIG. 6 shows a light conduit with irrigant channel in accordance with another implementation in this disclosure.

FIG. 7 shows a light conduit with irrigant channel in accordance with another implementation in this disclosure.

### DETAILED DESCRIPTION OF THE PREFERRED EMBODIMENTS

Embodiments regard selective sterilization of tissue using electromagnetic energy. The electromagnetic energy acts as a germicide. Infected or inflamed tissues (e.g., endodontic tissues or other internal tissues) can be treated using a chemo-mechanical debridement of canal spaces and closure of a canal opening. Some methods are available to further sterilize infected areas or initiate regeneration of local tissues. Embodiments regard light emitting diode (LED) treatment (e.g., at a specific wavelength 255 nm and 405 nm, among others) for the sterilization of internal tissues and the production of biomarkers related to tissue regeneration.

Antimicrobial effects of LED treatments on cultures of E. faecalis (E. faecalis) and the effects of LED treatment, in combination, on the production of osteoinductive, angiogenic, proliferative, and proinflammatory biomarkers from LED-treated HEPM cells and primary human gingival fibroblasts were determined. The LED treatment reduced the viability of E. faecalis. The LED treatment did not appreciably affect the viability of HEPM cells and human primary fibroblasts. The LED treatment at a first wavelength, alone or in combination with LED treatment at a second wavelength, of HEPM cells and human primary fibroblasts induced the production of biomarkers related to endodontic tissue regeneration. Embodiments provide a new treatment modality for the sterilization and regeneration of inflamed endodontic tissues using short periods of LED treatment.

Embodiments provide a new approach for disinfection of volumes of tissue, especially in dental applications with infected canals, especially the third of a canal (the deepest part of the infected tissue) that is likely not receiving a full effect of a chemical debridement (6% bleach solution), which in and of itself it is quite caustic if expressed past the canal of a tooth. Embodiments show that there is a synergistic approach between an irrigation solution to tissue disinfection and a light source treatment (the LED treatment). Embodiments indicate that future disinfection of the infected tissue can be accomplished with a lower concentration bleach solution with decreased risk to patients. Embodiments have been assessed with infected root canals. However, the devices and methods described herein include potential benefits throughout dental, medical, and perhaps industrial applications (e.g., food preparation, conditioning, sterilization or the like). The devices and methods described herein have shown positive results for bactericidal benefits (anti-microbial) and have also initiated the production of markers indicating proliferation (tissue regeneration).

Embodiments regard devices and methods for precisely applying electromagnetic energy for invasive disinfection (or potential regeneration) of a target volume of tissue. In some embodiments, these devices and methods are applied to a patient, such as a human being or other living organism, through an open incision, opening or the like. A delivery shaft assembly emits electromagnetic energy generated by a light element (or other electromagnetic energy generation element) of one or more specified wavelengths into/onto a targeted volume of tissue to reduce infectious tissue volume and prohibit the tissue from proliferating or to destroy existing infectious tissue. In some embodiments, application of 253 nanometer (nm) wavelength energy is used. Energy of this wavelength is sometimes called “germicidal ultraviolet light”. This specified wavelength has been proven to kill some bacteria. The effectiveness of the disclosed devices and methods can be a function of the amount of electromagnetic energy applied and the duration of application (e.g., the “time power product”). Embodiments regard application of electromagnetic energy to the target tissue both to prevent collateral damage to normal tissue, while also optimizing the efficiency of the claimed method. In the example application of 253 nm wavelength energy (known for deleterious effects on bacteria) a precision applicator is used. The precision applicator includes a delivery shaft selectively coupled (e.g., based on profile of the patient opening, positioning of the opening or the like) with a handle generator including the electromagnetic generating element. The disclosed devices can be used to apply the electromagnetic energy to infected tissue, through an incision or other opening in the subject. Further, the devices are equally effective when applied to a surface of a body of tissue (e.g., an abrasion or the like that is not internal to a patient).

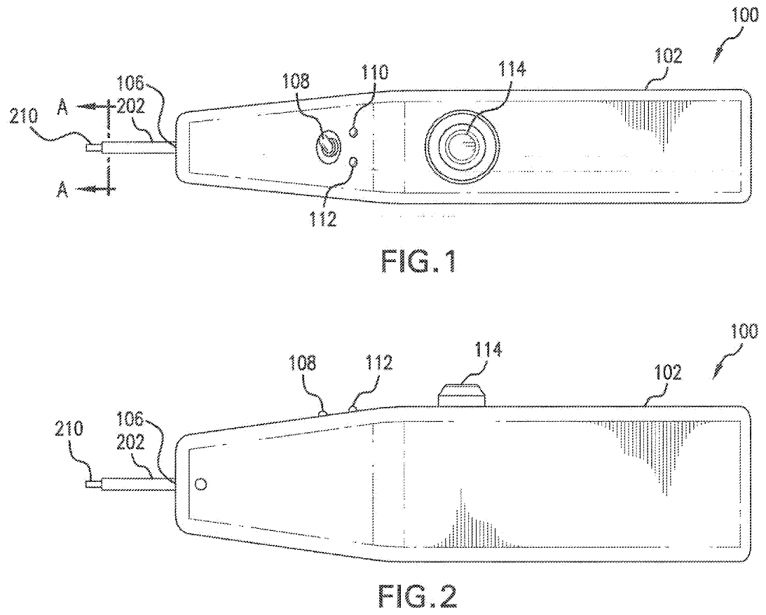

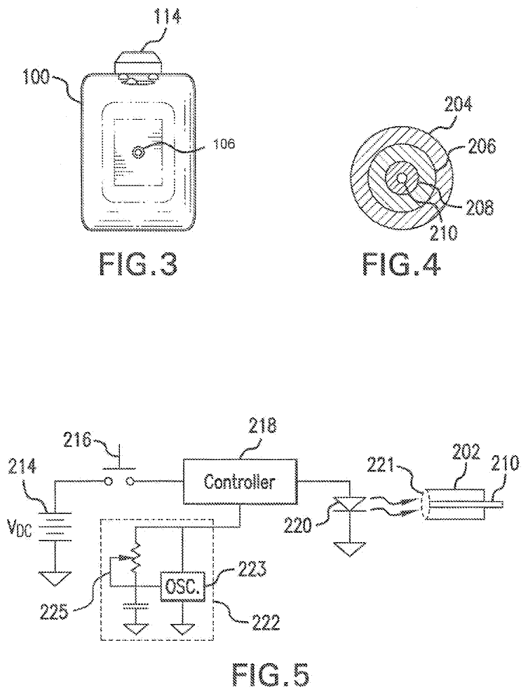

FIG. 1 illustrates, by way of example, a perspective view diagram of an embodiment of an electromagnetic energy-based tissue treatment system 100. FIG. 2 illustrates, by way of example, a front view diagram of an embodiment of the electromagnetic energy-based tissue treatment system 100. The system 100 as illustrated includes a generator housing 102, a light conduit 202, an attachment mechanism 106, a power toggle 108, indicator elements 110, 112, and an electromagnetic energy delivery switch 114.

One particular improvement of this particular electromagnetic energy-based tissue treatment system 100 can be found in light conduit 202. Light conduit 202 implemented as, for example, hollow tube, fiberoptic strand, etc., can be inserted into a cellular volume and positioned so as to apply light in the selected volume to treat an area of interest. One or more such light conduits can be moved about the volume so as to apply light power proportionally throughout the volume to achieve a particular stimulation.

Light conduit 202 in the form of a plastic fiber optic strand is disclosed. Section A-A shows light conduit 202. Light conduit 202 can comprise an outer jacket 204, surrounding a buffer layer 206, surrounding a cladding layer 208, surrounding a core 210. The end of light conduit 202 has all the outer layers removed leaving only core 210 to apply therapeutic light outward in a 3D volume. Removing cladding layer 208 is counter-intuitive to optical fiber applications, since cladding layer traps the light in the core so that it exits as a point source of light. With cladding layer removed toward the distal end of light conduit 202, therapeutic light sprays outward in three dimensions.

Light conduit 202 must also be made plastic or a non-thermal conductive material. A glass or metal light conduit, for example, in the form of fiberoptics conducts heat. It has been discovered that heat has a detrimental effect cellular structure. So, while UVC light has the microbial killing benefits discussed above to disinfect a wound, heat will damage the surrounding cells to interfere with the healing effect.

Light conduit 202 is preferably disposable and easily replaceable. There are many attachment mechanisms 106 for quickly attaching and removing light conduit 202 from the light source. Attachment mechanism 106 can be an optical fiber connector with cinch nut connectors at each end or any other form or compression or friction-hold connector or a bayonet connector that allows for simple insertion and quarter-rotation of light conduit 202 for a secure fit. Those skilled in the art will recognize that those skilled in the art of fiber optics are aware of many styles of attachment mechanisms 106 for light conduit 202 and that any of them that meet the design requirements are suitable for device 100.

Another particular improvement to device 100 can be found with reference to the electrical schematic for device 100, shown in FIG. 5. Device 100 contains a power source 214 in series with switch 114 for providing power to a controller 218 which drives a light source 220 that is illustrated as a UVC LED.

Controller 218 ensures a constant current to light source 220. As light source 220 heats up from the current flow, the resistance of light source 220 tends to decrease. A decrease in the resistance with a constant voltage drop causes current to increase. To prevent this thermal runaway, controller 218 maintains a constant current to light source 220 throughout the temperature range. Controller 218 can be implemented as a femtobucket, which is a constant current LED driver.

Controller 218 can also have a duty cycle control 222 that is manually adjustable by a rotating knob that adjusts the on/off time and pulsating rate in a manner. Duty cycle controller 222 varies the on/off time and pulsating rate of light source 220. Duty cycle controller 222 can comprise an oscillator 223 controlled by a variable resistive element 225. Oscillator 223 can be implemented as a variable frequency square wave generator, such as the 555 variable frequency oscillator. Variable resistive element 225 can be a rheostat or potentiometer.

In one implementation, duty cycle controller 120 can vary the on/off time of light source 110 and light source 112 from 1 HZ to 7.5 Hz that corresponds to 0.1 seconds on and 0.9 seconds off to 0.1 seconds on to 0.033 seconds off. Of course any range between these ranges or any other range of duty cycling the on/off time of light source 220 is applicable. The variation can be dependent upon a ratio that ameliorates or intensifies cellular healing or growth in the surrounding tissue vs. inhibition of microbes.

A power source 214 is provided to drive controller 218. In an embodiment, device 100 is powered by a dc power source, which can be in the form of multiple AA batteries. Power can be selectively applied to controller 218 by a switch 216. A switch 216 in the form of a push button prevents device from being inadvertently left on. Those skilled in the art will recognize, however, that any type of switch can be used.

A lens 221 is provided between light source 220 and the input of light conduit 202 for focusing the light from light source 220 into light conduit 202. Lens 221 reduces the amount of stray radiation into device 100 for more accurate determination of the amount of UVC radiation being applied to the treatment volume. It should also be understood that light source 220 may generate heat which has a negative effect on the circuitry as well as the therapeutic efficacy of device 100. Light conduit 221 serves to distance light source 220 from the application area, especially given that it is made of plastic or a non-thermal conductive material. Heat can also be moved away from light source 220 toward the handle of device 100 by one or more heatsinks.

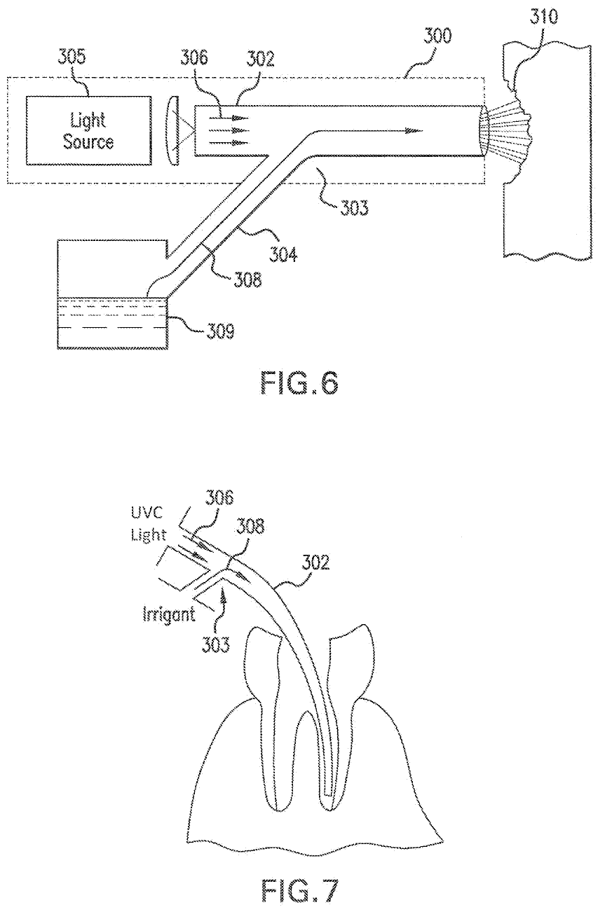

In another implementation, FIG. 6 shows a UVC light and irrigant device 300 comprising of a light conduit 302 with an irrigant channel 304. A light source 305 in accordance with the types discussed in the other embodiments herein is provided. Therapeutic and microbial killing UVC light 306 from light source 305 is combined with an irrigant 308 to provide enhanced antimicrobial effect. UVC light has a double acting effect of disinfecting irrigant 306 as it travels down light conduit 302 and disinfecting wound 310. This means less irrigant 308 can be used to treat wound 310 while simultaneously ensuring that irrigant 308 is disinfected before it comes into contact with wound 310.

Light source 305 is controlled by the circuitry similar to that already described in the alternative embodiments. Light conduit 302 can be the same as or similar to light conduit 202 discussed above. Light conduit 302 is provided with a branch 303 one of which is irrigant channel 304 that leads to irrigant reservoir 309. Irrigant 308 from irrigant reservoir can be fed into irrigant channel 304 by gravity, pressure, pumping, or any other manner. Irrigant could also be suctioned out of the volume through the same or different pump connected to branch 303. This would allow UVC activated irrigant to be washed into an infected volume and then suctioned out. A cover can be provided in irrigant channel 304 to stop or limit the flow of irrigant 308 into light conduit 302 or for suctioning irrigant out of the infected volume.

FIG. 7 shows another example with a light conduit 302 being used invasively, for example, in treating a root canal 315. Disinfectant UVC light 306 with disinfectant irrigant 308 can be simultaneously applied to the root canal 315. This gives the endodontist precise control over the application of the irrigant while allowing for the use of less irrigant. UVC light also activates the bleach by speeding up the oxidation process creating free radicals that rapidly react with organic compounds such as germs. In root canals, the irrigant is often bleach, which is highly damaging to tissue. If even the smallest amount of bleach is extruded into the tissues incredible pain and swelling can occur. By combining the UVC light with the irrigant, less irrigant or a more diluted irrigant can be used to clear out the infected area because the bleach is oxidized by the UVC light as it travels down light conduit 302 to the treatment area.

One skilled in the art will recognize that irrigant can be any form of irrigant, including, but not limited to, saline, bleach, water, etc. Such a device can be used for any external or invasive applications. Light conduit 202 can be narrow enough for invasive insertion into root canals, as shown in FIG. 10, or even into the body to treat internal infections.

Those skilled in the art will also recognize that device 300 can be housed in a portable device such as the embodiment shown in FIG. 1 or in equipment in a clinical setting.

The devices described herein emit electro-magnetic waves in the ultra-violet C spectrum to alter the structure of cells in the area of interest. The devices comprise of at least one light source for generating the light in the spectrum of interest, which preferably has a narrow-wavelength in the 200-280 nm range, including any range of values or any specific value within that range. Light source can be implemented as a light emitting diode, solid state laser, microwave generated UV plasma, or any type of fixed or tunable wavelength source.

While wavelength between 200 to 280 nm (inclusive). The selected wavelength of the light source may have a narrow spectral output centered around a specific wavelength of, for example, ±10 nm. A wavelength of 265 nm is generally accepted as the optimum as it is the peak of the DNA absorption curve as averaged for most germs, more specific wavelengths can be used to target specific germs. This means the light sources could have a mix of frequencies to specifically target a range of germs with each frequency selected for maximum absorption by the germs' DNA or could be modified with multiple light sources with different wavelengths.

Those skilled in the art will similarly recognize that the foregoing device can be used “invasively” through insertion into open tissue such as a knee joint, fistula, tumor, etc., with the device held firmly or slowly pulled through the volume of tissue for disinfecting the tissue as herein described.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention, which is not to be limited except by the following claims.

1. A device for disinfecting an area of interest, the device comprising:
an ultra-violet C spectrum (UVC) light source for emitting UVC light;
an irrigant source configured to supply an irrigant;
a light conduit in electromagnetic communication with the UVC light source configured to communicate light from the UVC light source to the area of interest;
an irrigant channel in communication with the irrigant source, the irrigant channel configured to communicate the irrigant from the irrigant source to the area of interest; and
a controller combined with the UVC light source for controlling the intensity of the light emitted to the area of interest.

2. The device of claim 1 wherein the irrigant channel is in communication with the light conduit allowing the UVC light and the irrigant to travel together down a portion of the light conduit before reaching the area of interest.

3. The device of claim 2 wherein the light disinfects the irrigant as the UVC light and irrigant travel together down the portion of the light conduit before reaching the area of interest.

4. The device of claim 2 wherein the light activates the irrigant by speeding up the oxidation process creating free radicals that rapidly react with organic compounds as the UVC light and irrigant travel together down the portion of the light conduit before reaching the area of interest.

5. The device of claim 1 further comprising a pump configured to force irrigant from the irrigant source to the irrigant channel.

6. The device of claim 1 further comprising a pump configured to suction irrigant from the area of interest into the light conduit.

7. The device of claim 1 further comprising a selectively operated cover configured to selectively prevent the flow of irrigant from the irrigant source into the light conduit.

8. The device of claim 1, wherein the irrigant is one of bleach, saline, and water.

9. The device of claim 1, wherein the light conduit comprises a cylindrical distal end and wherein light radiates from the cylindrical distal end in three dimensions.

10. The device of claim 1, wherein the light conduit is a plastic fiberoptic conduit comprising a cladding that has been removed from a distal end so that UVC light is emitted radially and axially relative to the distal end of the conduit for insertion into a volume of interest to treat the affected area of interest.

11. The device of claim 1, wherein the controller comprises of a constant current device for controlling the current to the UVC light source throughout the temperature range of the UVC light source.

12. The device of claim 1, and further comprising a DC battery power source inside the housing and configured to provide power to the UVC light source.

13. The device of claim 1, wherein the light source is a UVC light emitting diode configured to sterilize and regenerate inflamed endodontic tissues.

14. The device of claim 1, and further comprising: a lens, a housing for housing the UVC light source, the controller, and the lens; and an attachment mechanism for selectively attaching the light conduit to the housing with the light conduit aligned with the lens to focus light from the UVC light source into the light conduit.

15. The device of claim 1, and further comprising a heatsink positioned between the UVC light source and the light conduit, the heatsink configured to move heat away from the UVC light source toward the light conduit.

16. A device for disinfecting an area of interest, the device comprising:
a housing;
an ultra-violet C spectrum (UVC) light source for emitting UVC light;
an irrigant source configured to supply an irrigant;
a light conduit in electromagnetic communication with the UVC light source configured to communicate light from the UVC light source to the area of interest;
an attachment mechanism for selectively attaching the light conduit to the housing;
an irrigant channel in communication with the irrigant source, the irrigant channel configured to communicate the irrigant from the irrigant source to the area of interest;
a DC battery power source inside the housing and configured to provide power to the UVC light source;
a controller combined to the UVC light source for controlling the intensity of the light emitted to the area of interest; and
a heatsink positioned between the light source and the light conduit, the heatsink configured to move heat away from the UVC light source toward the light conduit.

17. A method of disinfecting an area of interest, the method comprising:
taking a device having an ultra-violet C spectrum (UVC) light source for emitting UVC light through a light conduit to the area of interest;
energizing the UVC light source;
inserting the light conduit into a patient's tooth so the UVC light sterilizes and regenerates inflamed endodontic tissues;
supplying an irrigant from an irrigant source through the light conduit so the UVC light and the irrigant travel together down a portion of the light conduit before reaching the area of interest;
activating the irrigant with the UVC light by oxidizing the irrigant to create free radicals that rapidly react with organic compounds.

18. The method of claim 17 further comprising using a controller to control the intensity of the light emitted to the area of interest.

19. The method of claim 17 further comprising suctioning the irrigant from the area of interest into the light conduit.

20. The method of claim 17 further comprising disinfecting the irrigant with the UVC light as the UVC light and irrigant travel together down the portion of the light conduit before reaching the area of interest.
